(19) United States
(12) Patent Application Publication
(10) Pub. No.: US 2019/0000943 A1
(43) Pub. Date: Jan. 3, 2019

(54) AAV6 VECTORS FOR IMMUNOTHERAPY

(71) Applicant: AAVET THERAPEUTICS, LLC, Alachua, FL (US)

(72) Inventor: George Vladimirovich ASLANIDI, Austin, MN (US)

(73) Assignee: AAVET THERAPEUTICS, LLC, Alachua, FL (US)

(21) Appl. No.: 15/752,370

(22) PCT Filed: Aug. 13, 2016
(86) PCT No.: PCT/US2016/046940

§ 371 Date: Feb. 13, 2018

Related U.S. Application Data
(60) Provisional application No. 62/204,950, filed on Aug. 13, 2015.

(51) Int. Cl. A61K 39/00; C12N 15/86; A61P 35/00

(52) U.S. Cl. CPC A61K 39/0011; C12N 15/86; A61K 2039/5256; C12N 2750/14143; A61P 35/00

(57) ABSTRACT

Provided herein are nucleic acids, recombinant adeno-associated virus (rAAV) particles, and compositions, as well as methods of use thereof for inducing immune responses, including protective immune responses for preventing or treating cancer. In some aspects, the rAAV particle includes a nucleic acid that expresses a cancer associated antigen. In some aspects, the rAAV particle is a rAAV particle having a mutation in a surface-exposed amino acid, such as tyrosine, threonine, or serine, that enhances transduction of dendritic cells.

### CROSS-REFERENCE TO RELATED APPLICATION

This application claims the benefit of priority to U.S. Provisional Patent Application Ser. No. 62/204,950, filed Aug. 13, 2015, the entire contents of which are incorporated herein by reference.

### BACKGROUND OF INVENTION

Although a naturally occurring anti-tumor immune response is detectable in patients, this response fails to control tumor growth. There are currently no effective techniques for delivering cancer-specific antigens to antigen presenting cells to promote or enhance a protective response in a subject.

### SUMMARY OF THE INVENTION

This application provides AAV-based compositions and methods for immunotherapy, for example to treat cancer. In some aspects, compositions and methods provide effective direct delivery of cancer-specific antigen to antigen-presenting cells in vivo in a subject (as opposed to delivering antigens to antigen-presenting cells ex vivo) in order to initiate or enhance a protective response (e.g., a protective immune response) in the subject. As described herein, rAAV vectors can be used to induce a protective response in a subject. Such vectors can be used for targeting a wide variety of human cancers.

In some embodiments, the application provides a recombinant adeno-associated virus (rAAV) particle comprising a nucleic acid that encodes a cancer-associated antigen under the control of a promoter. In some embodiments, the nucleic acid is an expression construct that is flanked on each side by an inverted terminal repeat sequence. In some embodiments, the cancer associated antigen is Prostatic Acid Phosphatase (PAP), Prostate specific antigen (PSA), Prostate-specific membrane antigen (PSMA), Cancer Antigen 15-3 (CA-15.3), Epidermal growth factor receptor 2 (Her2/neu), FMS-like tyrosine kinase 3 ligand (FLT3), Alpha-fetoprotein (AFP), Hepatocyte growth factor receptor (HGFR, c-Met), Glypican 3 (GLP3), Carcinoembryonic antigen (CEA), and/or Telomerase (TERT). In some embodiments, the nucleic acid is a single-stranded rAAV nucleic acid vector. In some embodiments, the nucleic acid is a double-stranded rAAV nucleic acid vector. In some embodiments, the nucleic acid is a self-complementary rAAV nucleic acid vector.

In some embodiments, the rAAV particle is an rAAV6 particle. In some embodiments, the rAAV6 particle comprises a modified capsid protein comprising a non-native amino acid substitution at a position that corresponds to a surface-exposed amino acid in a wild-type AAV6 capsid protein. In some embodiments, the non-native amino acid substitution is selected from a non-tyrosine amino acid at a wild-type tyrosine position, a non-serine amino acid at a wild-type serine position, a non-threonine amino acid at a wild-type threonine position, a non-lysine amino at a wild-type lysine position, or a combination thereof. In some embodiments, the rAAV6 capsid protein has a Valine at position 5663. In some embodiments, the rAAV6 capsid protein has a Valine at position T492. In some embodiments, the rAAV6 capsid protein has a Valine at position 5662 and a Valine at position T492.

In some embodiments, aspects of the application relate to compositions comprising an rAAV particle and a pharmaceutically acceptable carrier.

In some embodiments, an adjuvant is provided along with an rAAV particle. In some embodiments, the adjuvant is an unmethylated CpG oligodinucleotide, a granulocyte-macrophage colony-stimulating factor (GM-CSF), interleukin 12 (11-12), or an agonist of a toll-like receptor 9 (TLR9).

In some embodiments, aspects of the application provide an immunotherapeutic method for treating cancer in a subject by delivering an rAAV particle or composition described in the application to a subject in an amount sufficient to produce an immunotherapeutic response in the subject. In some embodiments, an adjuvant also is delivered to the subject. In some embodiments, a chemotherapeutic agent also is delivered to the subject. In some embodiments, an additional (e.g., secondary) immunotherapeutic agent also is delivered to the subject. Non-limiting examples of additional immunotherapeutic agents that can be administered in combination with the rAAV particles or compositions include a DNA plasmid vector containing (e.g., encoding) a cancer associated antigen, wherein the cancer associated antigen is the same as, similar to, or different than cancer associated antigen delivered by the rAAV particle; antibodies, and; PD1/PDL1 inhibitors. In some embodiments, one or more chemotherapeutic and/or immunotherapeutic agents can be administered before, during (e.g., in combination with), or after administration of the rAAV particle or composition.

In some embodiments, an rAAV particle or composition (e.g., alone or along with an adjuvant and/or a chemotherapeutic agent and/or an additional immunotherapeutic agent) is administered to the subject subcutaneously, intramuscularly, or intradermally.

In some embodiments, the subject was diagnosed as having cancer. In some embodiments, the subject is known to have an increased risk (relative to the average risk in a population) of developing cancer.

In some embodiments, the cancer is selected from the group consisting of lymphomas, hemangiosarcomas, mast cell tumors, osteosarcomas, melanomas, prostate cancer, thyroid cancer, liver cancer, pancreatic cancer, brain tumors, kidney cancer, ocular cancer, head or neck cancer, lung cancer, breast cancer, cervical cancer, gastrointestinal cancers, and urogenital cancers. As described in this application, these and other cancers can be treated by delivering one or more antigens characteristic of the target cancer directly to a subject (e.g., without delivering antigen presenting cells to the subject) using an rAAV.

In some embodiments, the subject is a mammal. In some embodiments, the subject is a human, a non-human primate, a companion animal, or a farm animal.

These and other aspects are described in more detail herein.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A, 1B, 1C and 1D show analysis of EGFP expression after transduction of mouse bone morrow-derived DCs with AAV6 capsid mutants. Surface-exposed serines (S) at position 663 and threonine (T) at position 492 were substituted with valine (V) and the mutant vectors here evaluated for their efficiency to mediate transgene expression. (FIG. 1A) shows an EGFP expression analysis at 48 hrs post-infection at an MOI of 2×10⁴ vgs/cell. (FIG. 1B) provides a quantitation of transduction efficiency of each of the mutant AAV6 vectors. Flow cytometry analysis of the number of EGFP positive cells is provided in (FIG. 1C) and mean fluorescence intensity in (FIG. 1D). *P<0.05,**P<0.01 vs. AAV6-WT.

FIGS. 2A, 2B, and 2C show phenotypic analysis of specific CD8⁺ cells induced by AAV6 vectors expressing OVA. C57BL/6 mice (n=3 per group) were i.m injected with AAV6-WT-OVA, AAV6-S663V+T492V-OVA and AAV6-EGFP. OVA-CD8⁺ cells were analyzed weekly in peripheral blood. (FIG. 2A) shows representative examples of OVA-CD8⁺ cells induced by different vectors at 2 weeks after injection. (FIG. 2B) provides a quantitation of the number of OVA-CD8⁺ cells by mutant AAV6 vectors. (FIG. 2C) shows a time course of OVA-CD8⁺ over 1, 2 and 3 weeks after vector administration, *P<0.05, **P<0.01 vs. AAV6-WT.

FIGS. 3A and 3B show an analysis of OVA-specific cytotoxic T-lymphocytes (CTLs) killing activity on RM1-OVA cells. (FIG. 3A) shows a Western blot analysis of the expression level of OVA (top blot) and PAP (middle blot) in murine myoblasts after delivery with AAV6 vectors. 2M is two mutations (S662V+T492V). Mouse prostate cancer cells and RM1-OVA served as positive control (first band in each blot). AAV-EGFP was used as negative control to eliminate the possibility of non-specific stimulation of gene expression. (FIG. 3B) illustrates results for CTLs generated from mice splenocytes after i.m. injection of AAV6-S662V+T492V and AAV6-WT vectors encoding OVA. AAV2-S662V+T492V-EGFP and AAV6-WT vectors were used to generate non-specific CTLs. A killing curve was generated with a decreasing number of effector cells and specific target cell lysis was determined by FACS analysis of live/dead cell ratios. *P<0.005 between the same capsid & a different gene, and ^{#}P<0.005 between a different capsid & the same gene, considered as significant.

FIGS. 4A and 4B show in vivo imaging of tumor growth progression evaluated by activity of luciferase stably expressed in murine prostate cancer cells, RM1. C57BL/6 mice were injected i.m. with 5×10e10 vgs/animal of the most efficient mutant AAV6 vectors carrying the prostatic acid phosphatase gene. Live images were taken weekly to analyze differences in luciferase activity for visual representation of the tumor size. The visual output represents the number of photons emitted/second/cm² as a false color image (FIG. 4A) and relative signal intensity (FIG. 4B). The life span of each animal challenged with cancer cells, *P<0.005 was considered as significant.

### DETAILED DESCRIPTION OF THE INVENTION

Provided herein are nucleic acids, recombinant adeno-associated virus (rAAV) particles, compositions, and methods for immunotherapy, for example for inducing or promoting a protective response in a subject in order to treat or assist in the treatment of a disease (for example cancer). In some embodiments, the treatment can prevent or slow the progression of the disease or disorder.

Aspects of the present application are related to the surprising effectiveness of rAAV vectors (for example, but not limited to, capsid-modified AAV6 vectors) for inducing a protective immune response in subjects, for example subjects having cancer. As described herein, rAAV vectors can be used to deliver cancer-specific antigens to a subject (e.g., to antigen presenting cells in the subject) to produce a protective response in the subject. In some aspects, rAAV vectors described herein provide a good balance of immunogenicity and high transduction efficiency for delivering cancer associated antigens to a subject in a therapeutically effective amount.

In some embodiments, rAAV vectors (e.g., capsid-modified AAV vectors) can transduce different subsets of dendritic cells, macrophages, progenitors cells such as monocytes (CD14+) or hematopoietic (CD34+) cells, or combinations thereof in a subject after direct administration to the subject (e.g., intradermally, subcutaneously, intramuscularly, or via any other suitable route as described in more detail herein).

In some embodiments, compositions described herein can be administered to subjects having cancer (e.g., diagnosed as having cancer) to treat or help treat the cancer (for example, alone or in conjunction with one or more additional anti-cancer therapies). In some embodiments, compositions described herein can be administered to prevent or help prevent the spread of a cancer or the further growth of a tumor. In some embodiments, one or more compositions described herein are administered to a subject as a vaccine for preventing formation of solid tumors and/or metastasis. In some embodiments, one or more compositions described herein can be administered to a subject post-surgery (or after other treatment), for example to reduce the risk or prevent recurrence of a cancer.

In some embodiments, compositions described herein can be administered as a vaccine to a subject (e.g., a subject at risk of cancer, for example due to one or more genetic risk factors, or due to exposure to one or more carcinogens and/or radiation) to reduce the risk or prevent the occurrence of a cancer.

Accordingly, in some embodiments aspects of the disclosure can be used for immunotherapy to treat one or more cancers. In some embodiments, rAAV compositions described herein may need to be administered to a subject more than once (for example to support an initial treatment by providing an immunotherapeutic boost at one or more later dates). In some embodiments, a different AAV serotype (or different capsid variants of an rAAV) are used for the different administrations. In some embodiments, 2-10 (e.g., 2, 3, 4, 5, 6, 7, 8 9, 10) or more administrations may be provided to a subject over a period of months to years (e.g., 1-12 months, 1-10, 10-25, 25-50, or more years).

In some embodiments, rAAV variants with increased efficiency of transducing nucleic acids into the nucleus of a target cell (e.g., as a result of reduced proteasomal degradation relative to wild-type AAV capsids) can be used.

In some embodiments, rAAV vectors described herein can promote mild inflammation that can promote maturation of target dendritic cells (or other target cells).

In some embodiments, an adjuvant can be administered to increase the effectiveness (e.g., therapeutic effectiveness) of an rAAV composition described herein. An adjuvant can be a pharmacological or immunological agent that modifies the effect of other agents. In some embodiments, an adjuvant can be a substance which enhances the body's immune response to an antigen. In some embodiments, an adjuvant is administered after initial treatment for cancer, especially to suppress secondary tumor formation. In some embodiments, the adjuvant is administered in combination with the rAAV particle or composition. In some embodiments, the adjuvant is administered as an additional immunotherapeutic agent.

In some embodiments, an adjuvant therapy can be provided to a subject in addition to treatment with an rAAV composition described herein. An adjuvant therapy can be an additional cancer treatment given after the primary treatment to lower the risk that the cancer will recur. An adjuvant therapy may include chemotherapy, radiation therapy, hormone therapy, targeted therapy, or biological therapy, or any combination of two or more thereof.

In some embodiments, adjuvants are added to (or administered in combination with) a vaccine (e.g., an rAAV composition) to modify the immune response by boosting it such as to give a higher amount of antibodies and a longer-lasting protection, thus minimizing the amount of injected foreign material (e.g., rAAV particles). In some embodiments, adjuvants may also be used to enhance the efficacy of a vaccine by helping to modify the immune response to particular types of immune system cells; for example, by activating the T cells instead of antibody-secreting B cells, depending on the purpose of the vaccine. Non-limiting examples of adjuvants include unmethylated CpG oligodinucleotide, a granulocyte-macrophage colony-stimulating factor (GM-CSF), interleukin 12 (11-12), an agonist of a toll-like receptor 9 (TLR9), aluminum hydroxide, paraffin oil, other adjuvants, or combinations of two or more thereof.

In some embodiments, one or more compositions described herein are administered along with an adjuvant, a chemotherapeutic drug, an additional immunotherapeutic agent, other cancer treatment (e.g., adjuvant therapy), or a combination of two or more thereof.

Recombinant Adeno-Associated Virus (rAAV) Particles and Nucleic Acids

Aspects of the disclosure relate to recombinant AAV (rAAV) particles and nucleic acids.

In some embodiments, a nucleic acid is provided, the nucleic acid comprising an expression construct containing a promoter (e.g., a truncated promoter) operably linked to a coding sequence of a gene of interest. In some embodiments, a promoter is a natural promoter. In some embodiments, a promoter can be a truncated natural promoter. In some embodiments, a promoter can include an enhancer and/or basal promoter elements from a natural promoter. In some embodiments, a promoter can be or include elements from a CMV, a chicken beta actin, a desmin, or any other suitable promoter or combination thereof. In some embodiments, a promoter can be an engineered promoter. In some embodiments, a promoter is transcriptionally active in dendritic cells. In some embodiments, a promoter is less than 1.6 kb in length, less than 1.5 kb in length, less than 1.4 kb in length, less than 1.3 kb in length, less than 1.2 kb in length, less than 1.1 kb in length, less than 1 kb in length, or less than 900 bp in length.

In some embodiments, an expression construct including a promoter and a gene of interest is flanked on each side by an inverted terminal repeat sequence (e.g., a naturally occurring or modified, AAV ITR).

The coding sequence of a gene of interest may be any coding sequence of any gene that is appropriate for use in immunotherapy. In some embodiments, the gene of interest is a gene that encodes a cancer associated antigen, for example a marker characteristic of a particular cancer. In some embodiments, the marker is unique to cancer cells (e.g., a mutant protein). In some embodiments, the marker is overexpressed in cancer cells relative to healthy cells. In some embodiments, the marker is a cell surface marker. Non-limiting example of genes of interest for treating prostate cancer as described herein include Prostatic Acid Phosphatase (PAP), Prostate specific antigen (PSA), and/or Prostate-specific membrane antigen (PSMA). Non-limiting example of genes of interest for treating breast cancer as described herein include Cancer Antigen 15-3 (CA-15.3), and/or Epidermal growth factor receptor 2 (Her2/neu). Non-limiting example of genes of interest for treating B cell lymphoma as described herein include FMS-like tyrosine kinase 3 ligand (FLT3). Non-limiting example of genes of interest for treating liver cancer include Alpha-fetoprotein (AFP), Hepatocyte growth factor receptor (HGFR, c-Met), and/or Glypican 3 (GLP3). Other non-limiting example of genes of interest for treating cancer include Carcinoembryonic antigen (CEA), and/or Telomerase (TERT). Other cancer markers can be used.

In some embodiments, the expression construct comprises one or more regions comprising a sequence that facilitates expression of the coding sequence of the gene of interest, e.g., expression control sequences operably linked to the coding sequence. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

In some embodiments, the nucleic acid is a plasmid (e.g., a circular nucleic acid comprising one or more of an origin of replication, a selectable marker, and a reporter gene). In some embodiments, a nucleic acid described herein, such as a plasmid, may also contain marker or reporter genes, e.g., LacZ or a fluorescent protein, and an origin of replication. In some embodiments, the plasmid is transfected into a producer cell that produces AAV particles containing the expression construct.

In some embodiments, the nucleic acid is a nucleic acid vector (e.g., a linear nucleic acid vector) such as a recombinant adeno-associated virus (rAAV) vector. Exemplary rAAV nucleic acid vectors useful according to the disclosure include single-stranded (ss) or self-complementary (sc) AAV nucleic acid vectors.

In some embodiments, a recombinant rAAV particle comprises a nucleic acid vector, such as a single-stranded (ss) or self-complementary (sc) AAV nucleic acid vector. In some embodiments, the nucleic acid vector contains an expression construct as described herein and one or more regions comprising inverted terminal repeat (ITR) sequences (e.g., wild-type ITR sequences or engineered ITR sequences) flanking the expression construct. In some embodiments, the nucleic acid is encapsidated by a viral capsid.

Accordingly, in some embodiments, a rAAV particle comprises a viral capsid and a nucleic acid vector as described herein, which is encapsidated by the viral capsid. In some embodiments, the viral capsid comprises 60 capsid protein subunits comprising VP1, VP2 and VP3. In some embodiments, the VP1, VP2, and VP3 subunits are present in the capsid at a ratio of approximately 1:1:10, respectively. In other embodiments, rAAV particles can have different numbers and ratios of VP1, VP2, and VP3 capsid proteins.

The ITR sequences of a nucleic acid or nucleic acid vector described herein can be derived from any AAV serotype (e.g., 1, 2, 3, 4, 5, 6, 7, 8, 9, or 10) or can be derived from more than one serotype. In some embodiments of the nucleic acid or nucleic acid vector provided herein, the ITR sequences are derived from AAV2. In some embodiments of the nucleic acid or nucleic acid vector provided herein, the ITR sequences are derived from AAV6. ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™ Viral Vectors for Gene Therapy Methods and Protocols. 10.1385/1-59259-304-6:201© Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the expression construct is no more than 7 kilobases, no more than 6 kilobases, no more than 5 kilobases, no more than 4 kilobases, or no more than 3 kilobases in size. In some embodiments, the expression construct is between 4 and 7 kilobases in size.

The rAAV particle may be of any AAV serotype (e.g., 1, 2, 3, 4, 5, 6, 7, 8, 9, or 10), including any derivative (including non-naturally occurring variants of a serotype) or pseudotype.

In some embodiments, the rAAV particle is an rAAV6 particle. In some embodiments, the rAAV particle is an rAAV2 particle. Non-limiting examples of derivatives and pseudotypes include AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.).

In some embodiments, the rAAV particle comprises a capsid that includes modified capsid proteins (e.g., capsid proteins comprising a modified VP3 region). Methods of producing modified capsid proteins are known in the art (see, e.g., U.S. Patent Publication Number US20130310443, which is incorporated herein by reference in its entirety). In some embodiments, the rAAV particle comprises a modified capsid protein comprising a (i.e., at least one) non-native amino acid substitution at a position that corresponds to a surface-exposed amino acid in a wild-type capsid protein (e.g., wild-type AAV6 capsid protein, such as SEQ ID NO: 1, wild-type AAV2 capsid protein, such as SEQ ID NO: 2, or other wild-type AAV capsid protein). In some embodiments, the rAAV particle comprises a modified capsid protein comprising a non-tyrosine amino acid (e.g., a phenylalanine) at a position that corresponds to a surface-exposed tyrosine amino acid in a wild-type capsid protein, a non-threonine amino acid (e.g., a valine) at a position that corresponds to a surface-exposed threonine amino acid in the wild-type capsid protein, a non-lysine amino acid (e.g., a glutamic acid) at a position that corresponds to a surface-exposed lysine amino acid in the wild-type capsid protein, a non-serine amino acid (e.g., a valine) at a position that corresponds to a surface-exposed serine amino acid in the wild-type capsid protein, or a combination thereof.

Exemplary surface-exposed amino acids include positions that correspond to S663, S551, Y705, Y731, and T492 of the wild-type AAV6 capsid protein. In some embodiments, a rAAV particle (e.g., a rAAV6 or other rAAV serotype particle) comprises a capsid that includes modified capsid proteins having one or more, for example two or more (e.g., 2, 3, 4, 5, or more) amino acid substitutions. Non-limiting examples of modified AAV6 capsid proteins include S663V+T492V, S663-551V, Y705-731F+T492V.

An exemplary, non-limiting wild-type AAV6 capsid protein sequence is provided below (SEQ ID NO: 1).

```
    1 MAADGYLPDW LEDNLSEGIR EWWDLKPGAP KPKANQQKQD DGRGLVLPGY
  51 KYLGPFNGLD KGEPVNAADA AALEHDKAYD QQLKAGDNPY LRYNHADAEF
101 QERLQEDTSF GGNLGRAVFQ AKKRVLEPFG LVEEGAKTAP GKKRPVEQSP
151 QEPDSSSGIG KTGQQPAKKR LNFGQTGDSE SVPDPQPLGE PPATPAAVGP
201 TTMASGGGAP MADNNEGADG VGNASGNWHC DSTWLGDRVI TTSTRTWALP
251 TYNNHLYKQI SSASTGASND NHYFGYSTPW GYFDFNRFHC HFSPRDWQRL
301 INNNWGFRPK RLNFKLFNIQ VKEVTTNDGV TTIANNLTST VQVFSDSEYQ
351 LPYVLGSAHQ GCLPPFPADV FMIPQYGYLT LNNGSQAVGR SSFYCLEYFP
401 SQMLRTGNNF TFSYTFEDVP FHSSYAHSQS LDRLMNPLID QYLYFLNRTQ
451 NQSGSAQNKD LLFSRGSPAG MSVQPKNWLP GPCYRQQRVS KTKTDNNNSN
501 FTWTGASKYN LNGRESIINP GTAMASHKDD KDKFFPMSGV MIFGKESAGA
551 SNTALDNVMI TDEEEIKATN PVATERFGTV AVNLQSSSTD PATGDVHVMG
601 ALPGMVWQDR DVYLQGPIWA KIPHTDGHFH PSPLMGGFGL KHPPPQILIK
651 NTPVPANPPA EFSATKFASF ITQYSTGQVS VEIEWELQKE NSKRWNPEVQ
701 YTSNYAKSAN VDFTVDNNGL YTEPRPIGTR YLTRPL
```

Exemplary surface-exposed tyrosine amino acids include positions that correspond to Y252, Y272, Y444, Y500, Y700, Y704, or Y730 of the wild-type AAV2 capsid protein. Exemplary surface-exposed serine amino acids include positions that correspond to S261, S264, S267, S276, S384, S458, S468, S492, S498, S578, S658, S662, S668, S707, or S721 of the wild-type AAV2 capsid protein. Exemplary surface-exposed threonine amino acids include positions that correspond to T251, T329, T330, T454, T455, T503, T550, T592, T581, T597, T491, T671, T659, T660, T701, T713, or T716 of the wild-type AAV2 capsid protein. Exemplary surface-exposed lysine amino acids include positions that correspond to K258, K321, K459, K490, K507, K527, K572, K532, K544, K549, K556, K649, K655, K665, or K706 of the wild-type AAV2 capsid protein. In some embodiments, a rAAV particle (e.g., a rAAV1, 2, 3, 4, 5, 6, 7, 8, 9, or 10 rAAV particle) comprises a capsid that includes modified capsid proteins having one or more, for example two or more (e.g., 2, 3, 4, 5, or more) surface-exposed amino acid substitutions at positions corresponding to one or more of the surface-exposed amino acids described for AAV2.

An exemplary, non-limiting wild-type AAV2 capsid protein sequence is provided below (SEQ ID NO: 2).

```
    1 MAADGYLPDW LEDTLSEGIR QWWKLKPGPP PPKPAERHKD DSRGLVLPGY
  51 KYLGPFNGLD KGEPVNEADA AALEHDKAYD RQLDSGDNPY LKYNHADAEF
101 QERLKEDTSF GGNLGRAVFQ AKKRVLEPLG LVEEPVKTAP GKKRPVEHSP
151 VEPDSSSGTG KAGQQPARKR LNFGQTGDAD SVPDPQPLGQ PPAAPSGLGT
201 NTMATGSGAP MADNNEGADG VGNSSGNWHC DSTWMGDRVI TTSTRTWALP
251 TYNNHLYKQI SSQSGASNDN HYFGYSTPWG YFDFNRFHCH FSPRDWQRLI
301 NNNWGFRPKR LNFKLFNIQV KEVTQNDGTT TIANNLTSTV QVFTDSEYQL
351 PYVLGSAHQG CLPPFPADVF MVPQYGYLTL NNGSQAVGRS SFYCLEYFPS
401 QMLRTGNNFT FSYTFEDVPF HSSYAHSQSL DRLMNPLIDQ YLYYLSRTNT
451 PSGTTTQSRL QFSQAGASDI RDQSRNWLPG PCYRQQRVSK TSADNNNSEY
501 SWTGATKYHL NGRDSLVNPG PAMASHKDDE EKFFPQSGVL IFGKQGSEKT
551 NVDIEKVMIT DEEEIRTTNP VATEQYGSVS TNLQRGNRQA ATADVNTQGV
601 LPGMVWQDRD VYLQGPIWAK IPHTDGHFHP SPLMGGFGLK HPPPQILIKN
651 TPVPANPSTT FSAAKFASFI TQYSTGQVSV EIEWELQKEN SKRWNPEIQY
701 TSNYNKSVNV DFTVDTNGVY SEPRPIGTRY LTRNL
```

Methods of producing rAAV particles and nucleic acid vectors are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, the nucleic acid vector (e.g., as a plasmid) may be combined with one or more helper plasmids, e.g., that contain a rep gene (e.g., encoding Rep78, Rep68, Rep52 and Rep40) and a cap gene (encoding VP1, VP2, and VP3), and transfected into a producer cell line such that the rAAV particle can be packaged and subsequently purified.

In some embodiments, the one or more helper plasmids includes a first helper plasmid comprising a rep gene and a cap gene and a second helper plasmid comprising other genes that assist in AAV production, such as a Ela gene, a E1b gene, a E4 gene, a E2a gene, and a VA gene. In some embodiments, the rep gene is a rep gene derived from AAV2 and the cap gene is derived from AAVS. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. Alternatively, in another example, Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known in the art or described herein, e.g., by iodixanol step gradient, CsCl gradient, chromatography, or polyethylene glycol (PEG) precipitation.

The disclosure also contemplates host cells that comprise at least one of the disclosed rAAV particles, expression constructs, or nucleic acid vectors. Such host cells include mammalian host cells, with human host cells being preferred, and may be either isolated, in cell or tissue culture. In the case of genetically modified animal models (e.g., a mouse), the transformed host cells may be comprised within the body of a non-human animal itself.

Compositions

Aspects of the disclosure relate to compositions comprising rAAV particles or nucleic acids described herein. In some embodiments, rAAV particles described herein are added to a composition, e.g., a pharmaceutical composition.

In some embodiments, the composition comprises a pharmaceutically acceptable carrier. The term “carrier” refers to a diluent, adjuvant, excipient, or vehicle with which the rAAV particle is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases). Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of rAAV particles to human subjects. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice of Pharmacy, 22nd edition, Pharmaceutical Press, 2012.

Typically, such compositions may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared ins such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, a composition described herein may be administered to a subject in need thereof, such as a subject having a cancer. In some embodiments, a method described herein may comprise administering a composition comprising rAAV particles as described herein to a subject in need thereof. In some embodiments, the subject is a human subject. In some embodiments, the subject has or is suspected of having a disease that may be treated with immunotherapy, such as cancer. In some embodiments, the subject has been diagnosed with cancer. In some embodiments, the subject is known to be at risk of having or developing cancer.

In some embodiments, a composition also comprises one or more adjuvants. Non-limiting examples of adjuvants include one or more unmethylated CpG oligodinucleotides, granulocyte-macrophage colony-stimulating factor (GM-CSF), interleukin 12 (11-12), agonists of toll-like receptors 9 (TLR9), or any other suitable adjuvant or any combination of two or more thereof. However, in some embodiments, one or more adjuvants may be provided in a separate composition than an rAAV particle and/or nucleic acid composition described herein. In some embodiments, an adjuvant composition may be administered along with (e.g., simultaneously or concurrently with) an rAAV particle and/or nucleic acid composition described herein.

In some embodiments, a composition also comprises one or more chemotherapeutic or other anti-cancer agents (e.g., cytotoxic compounds, therapeutic antibodies, or other agents). However, in some embodiments, one or more anti-cancer agents may be provided in a separate composition than an rAAV particle and/or nucleic acid composition described herein. In some embodiments, an anti-cancer agent may be administered along with (e.g., simultaneously or concurrently with) an rAAV particle and/or nucleic acid composition described herein.

In some embodiments, a composition also comprises an additional immunotherapeutic agent (e.g., a DNA plasmid vector encoding a cancer associated antigen, wherein the cancer associated antigen is the same as, similar to or different than the cancer associated antigen delivered by the rAAV; antibodies; PD1/PDL1 inhibitors; and/or other immunotherapeutic agents). However, in some embodiments, one or more additional immunotherapeutic agents may be provided in a separate composition than an rAAV particle and/or nucleic acid composition described herein. In some embodiments, an additional immunotherapeutic composition may be administered along with (e.g., simultaneously or concurrently with) an rAAV particle and/or nucleic acid composition described herein.

Methods

Aspects of the disclosure relate to methods of delivering a nucleic acid (e.g., in an rAAV particle described herein) to a subject in order to induce an immune response, for example a protective immune response. In some embodiments, a composition described herein is administered to a subject at risk for cancer or having cancer (e.g., a subject diagnosed with cancer).

In some embodiments, the method comprises administering a rAAV particle as described herein or a composition as described herein to a subject via a suitable route to promote an immune response.

In some embodiments, a subject is a mammal. In some embodiments, a subject is a human subject. In some embodiments, the subject is a non-human primate. In some embodiments, a subjects is a companion animal (e.g., a dog, a cat, or other companion animal). In some embodiments, a subject is a farm animal (e.g., a horse, cow, sheep, or other farm animal). However, aspects of the disclosure can be used to treat other animals (e.g., other mammals).

Accordingly, aspects of the disclosure relate to methods of treating cancer. In some embodiments, the method comprises administering a therapeutically effective amount of an rAAV particle or a composition as described herein to a subject having cancer.

Non-limiting examples of cancer that can be treated according to methods described herein include lymphoma, hemangiosarcoma, mast cell tumors, breast cancer, osteosarcoma, melanoma, prostate cancer, thyroid cancer, liver cancer, kidney cancer, ocular cancer, head or neck cancer, lung cancer, gastrointestinal cancers (e.g., stomach, colon, or other gastrointestinal cancer), urogenital cancers, or cancers of other tissues or organs.

In some embodiments, an rAAV composition described herein (e.g., a mutant AAV6) is used to deliver a gene that expresses a suitable target for immunotherapy, for example an antigen or epitope that is characteristic of a cancer being treated. In some embodiments, the antigen or epitope is a marker that is unique to the cancer of interest. In some embodiments, the antigen or epitope is a marker that is over-expressed in the cancer of interest. Non-limiting examples of targets for immunotherapy include prostatic acid phosphatase for prostate cancer and FMS-like tyrosine kinase 3 ligand for B cell lymphoma. However, other proteins or peptides can be delivered for immunotherapy as described herein.

To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host organ, tissue, or cell (e.g., dendritic cells, macrophages, progenitor cells, for example a CD14+ monocytes, or CD34+ hematopoietic cells, or combinations thereof).

A therapeutically acceptable amount may be an amount that is capable of treating a disease, e.g., a cancer, by stimulating an immune response that can help treat the disease (e.g., alone or in combination with one or more additional anti-cancer therapies such as chemotherapy, additional immunotherapy (e.g., secondary immunotherapy), monoclonal antibody treatment, hormonal treatment, radiation, surgery or other treatment).

As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

The rAAV particle or nucleic acid vector may be delivered in the form of a composition, such as a composition comprising the active ingredient, such as a rAAV particle described herein, and a pharmaceutically acceptable carrier as described herein. The rAAV particles or nucleic acid vectors may be prepared in a variety of compositions, and may also be formulated in appropriate pharmaceutical vehicles for administration to human or animal subjects.

In some embodiments, the number of rAAV particles administered to a subject may be provided in a composition having a concentration on the order ranging from 10⁶ to 10¹⁴ particles/ml or 10³ to 10¹⁵ particles/ml, or any values therebetween for either range, such as for example, about 10⁶, 10⁷, 10⁸, 10⁹, 10¹⁰, 10¹¹, 10¹², 10¹³, or 10¹⁴ particles/ml. In one embodiment, rAAV particles of higher than 10¹³ particles/ml are administered. In some embodiments, the number of rAAV particles administered to a subject may be on the order ranging from 10⁶ to 10¹⁴ vector genomes(vgs)/ml or 10³ to 10¹⁵ vgs/ml, or any values therebetween for either range, such as for example, about 10⁶, 10⁷, 10⁸, 10⁹, 10¹⁰, 10¹¹, 10¹², 10¹³, or 10¹⁴ vgs/ml. In one embodiment, rAAV particles of higher than 10¹³ vgs/ml are administered. The rAAV particles can be administered as a single dose, or divided into two or more administrations as may be required to achieve therapy of the particular disease or disorder being treated. In some embodiments, 0.0001 ml to 10 mls are delivered to a subject. In some embodiments, the number of rAAV particles administered to a subject may be on the order ranging from 10⁶-10¹⁴ vg/kg, or any values therebetween, such as for example, about 10⁶, 10⁷, 10⁸, 10⁹, 10¹⁰, 10¹¹, 10¹², 10¹³, or 10¹⁴ vgs/kg. In some embodiments, the number of rAAV particles administered to a subject may be on the order ranging from 10¹²-10¹⁴ vgs/kg.

If desired, rAAV particles may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles may thus be delivered along with various other agents as required in the particular instance. Such compositions may be purified from host cells or other biological sources, or alternatively may be chemically synthesized. In some embodiments, the rAAV are delivered along with one or more adjuvants and/or one or more chemotherapeutic agents and/or one or more additional immunotherapeutic agents.

In some embodiments, the agent is an immunotherapeutic agent. The immunotherapeutic agent can be administered before, during or after administration of the rAAV particle or composition. In some embodiments, an additional immunotherapeutic composition is administered with the rAAV particle or composition. An “additional immunotherapeutic composition” is an immunotherapeutic composition that is administered before, during, or after administration of the rAAV particle or composition, which aids in the reduction of the frequency or severity of at least one sign or symptom of a disease or disorder experienced by a subject. Non-limiting examples of immunotherapeutic agents that can be administered in combination with the rAAV particles or compositions include a DNA plasmid vector containing a cancer associated antigen, wherein the cancer associated antigen is the same as, similar to or different than the cancer associated antigen; antibodies, and; PD1/PDL1 inhibitors (e.g., nivolumab, pembrolizumab, atezolizumab, or MEDI4736), other suitable immunotherapeutic agents, or combinations of two or more thereof. In some embodiments, the additional immunotherapeutic agent can be administered before, during or after administration of the rAAV particle or composition.

In some embodiments, the rAAV particle or composition is useful to treat prostate cancer, lymphoma, breast cancer, melanoma, or osteosarcoma. In some embodiments, the rAAV particle or composition is useful to treat a different cancer or several different cancers.

In some embodiments, the rAAV particle or composition is administered in parallel with an additional treatment. The rAAV particle or composition can be administered before, during or after administration of the additional treatment. In some embodiments, the additional treatment is administered to treat the same disorder or disease that is targeted by the rAAV particle.

In certain circumstances it will be desirable to deliver rAAV particles in suitably formulated pharmaceutical compositions disclosed herein via a route that stimulates an immune response. In some embodiments, rAAV particles are delivered to the dermis or epidermis of a skin of a subject. In some embodiments, rAAV particles are delivered into a muscle of a subject. Accordingly, rAAV particles may be delivered via an intradermal, subcutaneous, and/or intramuscular injection. However, other routes of administration may be used. In some embodiments, rAAV particles are delivered to the foot pad of an animal. In some embodiments, rAAV particles are delivered by injection to one or more other tissues or organs in an amount sufficient to induce an immune response (for example a protective immune response). In some embodiments, rAAV particle are delivered topically, orally, by injection, by inhalation (e.g., by nasal inhalation), or a combination thereof. In some embodiments, rAAV particles are delivered intraocularly, intravitreally, subretinally, parenterally, intravenously, intracerebro-ventricularly, or intrathecally.

In some embodiments, rAAV particles are not delivered systemically, for example to avoid expression in the liver or other site that could produce tolerance as opposed to an immune response.

The pharmaceutical forms of the rAAV particle compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subretinal, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles in the required amount in the appropriate solvent with several of the other ingredients enumerated above, as required, followed by filtered sterilization or another sterilization technique. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The amount of rAAV particle or nucleic acid vector compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle compositions, either over a relatively short, or a relatively prolonged period of time, as may be determined by the medical practitioner overseeing the administration of such compositions.

The composition may include rAAV particles, either alone, or in combination with one or more additional active ingredients, which may be obtained from natural or recombinant sources or chemically synthesized.

Toxicity and efficacy of the compositions utilized in methods of the disclosure can be determined by standard pharmaceutical procedures, using either cells in culture or experimental animals to determine the LD50 (the dose lethal to 50% of the population). The dose ratio between toxicity and efficacy is the therapeutic index and it can be expressed as the ratio LD50/ED50. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED50 with little or no toxicity. The dosage may vary within this range depending upon the dosage form employed and the route of administration utilized.

Subjects

Aspects of the disclosure relate to methods for use with a subject, such as human or non-human primate subjects. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals (e.g., companion animals) such as dogs and cats; livestock such as horses, cattle, pigs, sheep, goats, and chickens; and other animals such as mice, rats, guinea pigs, and hamsters.

In some embodiments, the subject has or is suspected of having a disease that may be treated with immunotherapy (e.g., alone or in combination with additional anti-cancer therapy). In some embodiments, the subject has or is suspected of having cancer. Subjects having cancer can be identified by a skilled medical practitioner using methods known in the art, e.g., by measuring serum concentrations of cancer-associated markers, genetic analysis, CT, PET, or MRI scans, tissue biopsies, or any combination thereof.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1. Reprogramming Immune Response with Capsid-Optimized AAV6 Vectors for Immunotherapy of Cancer

In the current studies novel capsid-optimized AAV serotype 6 (AAV6) vectors expressing a tumor-associated antigen were generated, and assessed for their ability to activate a protective T-cell response in an animal model. First, it was shown that specific mutations in the AAV6 capsid increase the transduction efficiency of these vectors in mouse bone marrow-derived dendritic cells in vitro approximately 5-fold compared with the wild-type (WT) AAV6 vectors. Next, the ability of the mutant AAV6 vectors to initiate specific T-cell clone proliferation in vivo was evaluated. The data indicate that the intramuscular administration of AAV6-S663V+T492V vectors expressing ovalbumin (OVA) led to a strong activation (approximately 9%) of specific T-cells in peripheral blood compared to AAV6-WT treated animals (less than 1%). These OVA-specific T-cells have a superior killing ability against mouse prostate cancer cell line RM1 stably expressing the OVA antigen when propagated in vitro. Finally, the ability of capsid-optimized AAV6-S663V+T492V vectors to initiate a protective anti-cancer immune response in vivo was evaluated. The results document the suppression of subcutaneous tumor growth in animals immunized with AAV6-S663V+T492V vectors expressing prostatic acid phosphatase (PAP) for approximately four weeks in comparison to one week and two weeks for the negative controls, AAV6-EGFP and AAV6-WT-PAP treated mice, respectively.

These studies suggest that successful inhibition of tumor growth in an animal model sets the stage for clinical applications of the capsid-optimized AAV6-S663V+T492V vectors.

Although a naturally occurring anti-tumor immune response is detectable in patients, this response fails to control tumor growth. The possibility of stimulating a specific anti-tumor immune response via genetically-modified dendritic cells (DCs) in both ex vivo and in vivo protocols, has been proven in a number of clinical trials (1-4). However, current methods for therapeutic antigen delivery, control of expression, proper mobilization of antigen presenting cells and, thus presentation of epitopes to effector cells, are not sufficient (3, 5). The common methods of antigen delivery include naked or lipid encapsulated DNA/RNA, peptides or recombinant proteins. The safety of these methods of delivery comes with a price of poor immunogenicity. In contrast, viral vectors are often highly immunogenic, and they also carry the risk of pathogenesis. For example, the major advantages of using adenovirus (Ad) vectors as a vaccine platform include their ability to infect a broad range of hosts and to induce high levels of transgene expression. However, infection with Ad vectors up-regulate co-stimulatory molecules accompanied by increase in proinflammatory cytokine and chemokine production by DCs. This early stimulation of DCs can contribute to more of an effective presentation of virus-derived epitopes rather than epitopes from recombinant antigens. In contrast, vaccinia virus-based vectors suppress maturation on antigen presenting cells, and thus impart the ability of DCs to properly stimulate specific T-cell clone proliferation (5-9).

According to aspects of the present disclosure, vectors based on adeno-associated virus (AAV) have superior transduction efficiency in broad cell types and a lack of pathogenicity (10-13). AAV vector-based antigen delivery to different subsets of DCs has been utilized successfully (14-19). These vectors have also been used for both passive and active immunization strategies (20-26).

The efficacy of wild-type (WT) AAV vectors can be significantly enhanced by substituting critical serine (S) and threonine (T) residues on their capsids to valine (V). These residues were identified by analysis of the AAV capsid crystal structure and they can be recognized and phosphorylated by common serine/threonine cellular kinases such as JNK and p38 MAPK (14). Several different amino acids were tested and (V) was chosen because of the similarity of its structure with both (S) and (T), and lack of recognition by kinases. Thus, these modifications can prevent kinase-mediated phosphorylation of the AAV capsid, and subsequent ubiquitination and proteasome-mediated degradation of the vectors (14, 27-29). These studies have led to the development of a number of AAV serotype 2 (AAV2) and serotype 6 (AAV6) vectors with high activity in human monocyte-derived dendritic cells (moDCs) (14, 15, 18).

In the present studies, the possibility of using capsid-optimized AAV6 vectors for active immunization against prostate cancer in vivo was evaluated. Subcutaneously injected mouse prostate carcinoma cell line RM1 (from Ras+Myc transformed C57BL/6 mouse) stably expressing firefly luciferase (Fluc) for the visual presentation of tumor size was used as an animal model. The following observations have been documented: (i) Site-directed mutagenesis of the surface-exposed (S) and (T) residues on the AAV6 capsid to (V) significantly improves transduction efficiency of S663V+T492V mutant compared with the AAV6-WT vectors in mouse bone morrow-derived DCs; (ii) Intramuscular injection of AAV6-S663V+T492V vectors expressing ovalbumin (OVA) leads to specific OVA-CD8⁺ cell proliferation with a higher number in peripheral blood two weeks after injection; (iii) Specific CD8⁺ cells generated by AAV6-S663V+T492V vectors can be expanded in vitro and show an increased killing ability when compared with cells generated by AAV6-WT vectors; (iv) Vaccination with AAV6-S663V+T492V vectors encoding the prostatic acid phosphatase (PAP) gene leads to a significant delay in prostate cancer progression and extends life span in a mouse model. These observations suggest that vaccination with capsid-modified AAV6 vectors against cancer is feasible, which supports the use of these vectors as a platform for in vivo vaccination.

Cells.

The mouse prostate carcinoma cell line RM1 and RM1 stably expressing OVA protein (RM1-OVA) were maintained as monolayer cultures in DMEM (Invitrogen) supplemented with 10% FBS (Sigma) and antibiotics (Lonza). The cell line was derived from a heterogeneous primary tumor in the prostate of a Ras-and-Myc transformed C57BL/6 mouse and genetically modified for stable OVA expression under the control of the strong CMV promoter. A RM1 cell line was modified to stably express a firefly luciferase (FLuc) driven by a CMV promoter for the monitoring of progression or reduction of the tumor, using previously described methods (48). RM1-FLuc cells are tumorigenic when grafted subcutaneously into syngeneic C57BL/6 hosts. Mouse conventional DCs were differentiated from bone morrow derived CD34⁺ cells in the presence of mrGM-CSF (2000 U/ml) and mrlL-4 (1000 U/ml) for 7 days. Briefly, marrow from 6 wk old C57BL/6 male mice was harvested by flushing with 1 ml PBS/bone. Cells were pelleted by centrifugation and contamination with red blood cells was cleaned with ACK lysis buffer at room temp for 5 min. Cells were purified with magnetic MicroBeads labeled with CD117 antibodies by loading suspension into a MACS Column which was placed in the magnetic field of a MACS Separator (Miltenyi Boitec). Prior to rAAV6 transduction, cells were characterized for co-stimulatory molecules expression to ensure that they met the typical phenotype of dendritic cells (DCs) (CD11c-RPE, Invitrogen).

Production of Recombinant AAV Vectors.

Site-directed mutagenesis was performed with plasmid pACGr2c6 as described previously (14, 15). Recombinant AAV vectors containing the EGFP, OVA and PAP genes driven by the chicken β-actin promoter were generated in HEK293 cells transfected with Polyethylenimine (PEI, linear, MW 25,000, PolySciences, Inc.). Vectors were purified by iodixanol (Sigma) gradient centrifugation and ion exchange column chromatography (HiTrap Sp Hp 5 ml, GE Healthcare). Virus was then concentrated and the buffer exchanged in three cycles to lactated Ringer's using centrifugal spin concentrators (Apollo, 150-kDa cut-off, 20-ml capacity, CLP). DNase I-resistant AAV particle titers were determined by RT-PCR with the following primer-pair, specific for the CBA promoter: forward 5′-tcccatagtaacgccaatagg-3′ (SEQ ID NO: 3), reverse 5′-CTTGGCATATGATACACTTGATG-3′ (SEQ ID NO: 4) and SYBR Green PCR Master Mix (Invitrogen) (14, 15, 49).

Recombinant AAV Vector Transduction Assays In Vitro.

Bone morrow-derived mouse DCs, were transduced with AAV6 vectors with 20,000 vgs/cell or and incubated for 48 hrs. Transgene expression was assessed as the total area of green fluorescence (pixel²) per visual field (mean±SD) as described previously (14, 15, 27). Analysis of variance was used to compare test results and the control, which were determined to be statistically significant.

Antibodies.

Western blotting was performed as described previously (14, 15) with following antibodies: anti-PAP (Fitzgerald, 1:1000); anti-OVA (Thermo Scientific Pierce, 1:1000) and b-actin (CellSignaling, 1:2000). Antibodies for FACS analysis were obtained as follow Anti-Human/Mouse CD45R (B220)-APC-A, CD19-APC-Cy7-A and CD8-V450-A (BD Biosciences); MHC Class I Murine Tetramer (Beckman Coulter).

Specific Cytotoxic T-Lymphocytes Generation and Cytotoxicity Assay.

Ten weeks old C57BL/6 male mice were injected i.m. with AAV6-WT-OVA, AAV-S663V+T492V-OVA and AAV6-WT-GFP. Spleens were harvested 2 weeks after and OVA-CD8⁺ cells were expanded in vitro in RPMI-1640 medium, supplemented with predominant for C57BL/6 mice OVA-derived SIINFEKL (SEQ ID NO: 5) peptide (10 ug/ml) (AnaSpec), rmlL-15 (10 ng/ml) and rmlL-21(25 ng/ml). Fresh supplements were added every 2 days. Stimulated T-cells were used for a killing assay against mouse prostate cell line RM1 stably expressing OVA. A killing curve was generated and specific cell lysis was determined by FACS analysis of live/dead cell. The target cells were pre-stained with 3,3-dioctadecyloxacarbocyanine (DiOC18(3)), a green fluorescent membrane stain, lx105 target RM1-OVA cells were co-cultured overnight with different ratios of CTLs (80:1, 50:1, 20:1, 10:1, 5:1). Membrane-permeable nucleic acid counter-stain, propidium iodide, was added to label the cells with compromised plasma membranes. Percentages of killed, double-stain positive cells were analyzed by flow cytometry (14, 15, 50).

Evaluation of the Tumor Growth by In Vivo Bioluminescence Imaging.

C57BL/6 male mice (Jackson Laboratory, Bar Harbor, Me.) were used for animal studies. Ten-week-old C57BL/6 male mice were injected intramuscularly with 5×10e10 vgs/animal of AAV6-WT-PAP, AAV6-S663V+T492V-PAP and AAV6-WT-GFP. Two weeks later RM1-FLuc cancer cells were injected subcutaneously. Luciferase activity was analyzed every week after injection using a Xenogen IVIS Lumina System (Caliper Life Sciences). Briefly, mice were anesthetized with 2% isoflurane and injected intraperitoneally with luciferin substrate (Beetle luciferin, Caliper Life Sciences) at a dose of 150 ug/g of body weight. Mice were placed in a light-tight chamber and images were collected at 5 minutes after the substrate injection. Images were analyzed by the Living Image 3.2 software (Caliper Life Sciences) to determine relative signal intensity(27). All animal experiments were approved by the University of Florida Institutional Animal Care and Use Committee. All procedures were done in accordance with the principles of the National Research Council's Guide for the Care and Use of Laboratory Animals. All efforts were made to minimize suffering of the animals challenged with cancer cells. Animals were monitored daily and humanely euthanized when tumor reached 0.5 cm in diameter.

Statistical Analysis.

All results are presented as mean±S.D. Differences between groups were identified using a grouped-unpaired two-tailed distribution of Student's T-test. P-values <0.05 were considered statistically significant.

Site-directed mutagenesis of surface-exposed serine (S) and threonine (T) residues on AAV6 capsid improves vector-mediated trans gene expression.

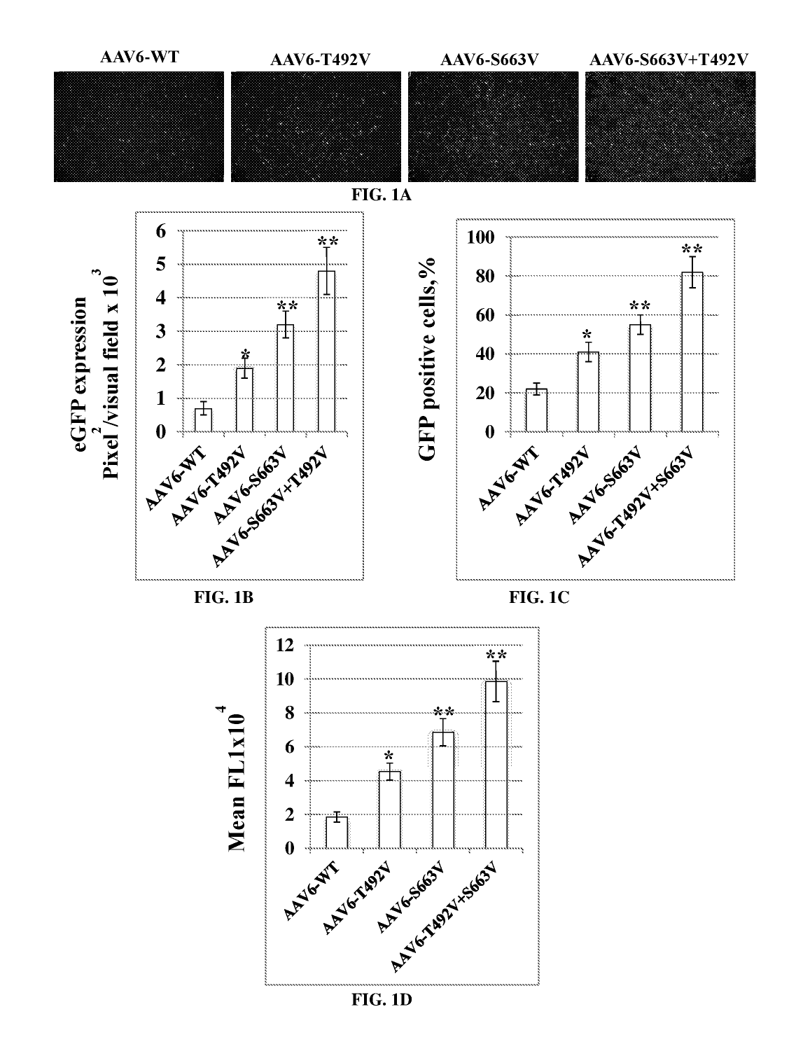

The AAV6 capsid contains 17 serine (S) and 15 threonine (T) surface-exposed residues in the viral protein 3 (VP3) common regions. It was previously showed that mutations of the single critical serine at position 663 and threonine at position 492 to valine (V) increased the transduction efficiency of the AAV6 vectors in human moDCs. Moreover, a combination of these mutations on the same viral capsid (S663V+T492V) further improved the transduction efficiency (15). In the current studies, evaluation of whether a similar approach could be used to increase the activity of AAV6 vectors in mouse bone morrow-derived DCs was performed. These results generated as total area of fluorescence/per visual field (FIGS. 1A and 1B) indicate that the AAV6-T492V-EGFP and AAV6-S663V-EGFP mutants transduced mouse DCs 2-fold and 3-fold more efficiently than their WT counterpart. Similar to previous observations in human DCs, a combination of two single mutations had an additive effect, since transduction efficiency of double-mutant AAV6-S663V+T492V-EGFP was increased to ˜5-fold compared with AAV6-WT. These results were confirmed by flow cytometry analysis and suggest that both, number of infected cells and fluorescence intensity (expression level), were increased by AAV6-S663V+T492V over AAV6-WT for ˜4-fold and ˜5-fold correspondingly (FIGS. 1C and 1D). The data suggest that the capsid-optimized AAV6 vectors can be used to achieve a high level of expression in mouse antigen presenting cells.

Capsid-Optimized AAV6 Vectors can Stimulate Specific T-Cell Clone Proliferation In Vivo.

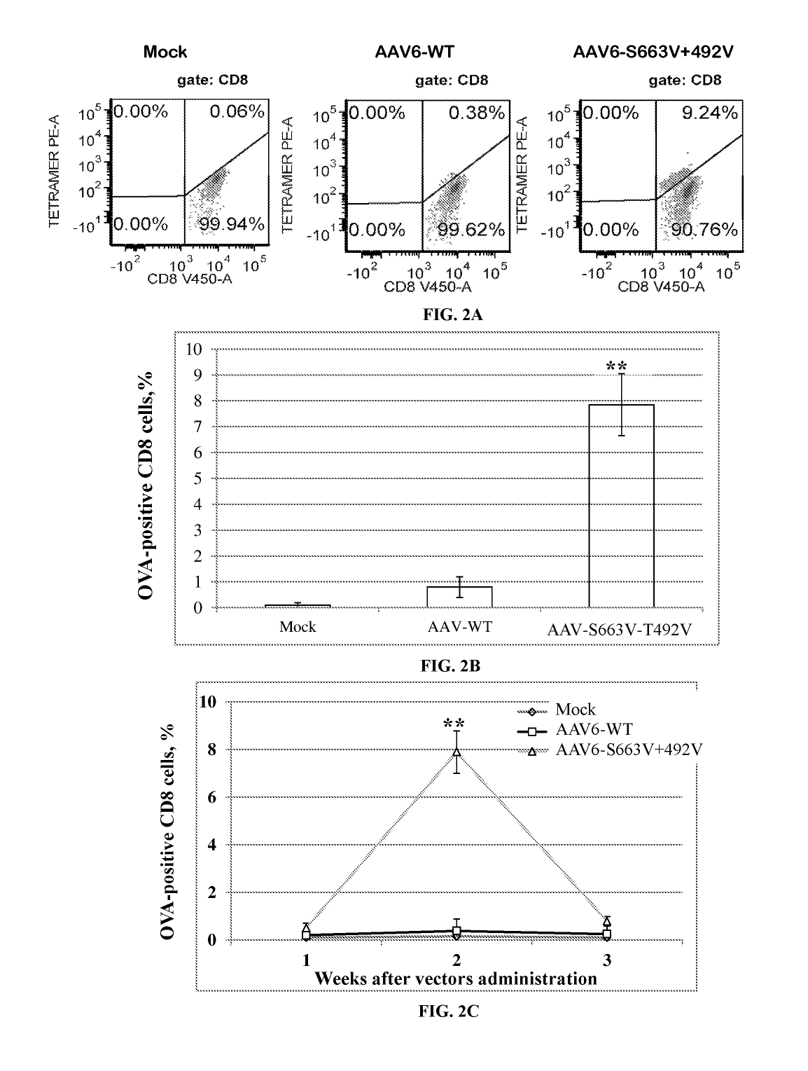

In the current studies, the possibility of using capsid-optimized AAV6 vectors to express antigens in mouse antigen presenting cells and to activate a specific T-cell proliferation in vivo was evaluated. Ovalbumin (OVA), commonly used as an immunogen, delivered by capsid-optimized AAV6-S663V+T492V or WT vectors was used to evaluate the immune responses. C57BL/6 mice were injected intramuscularly (i.m.) at various time points post viral injection. Numbers of OVA-specific CD8⁺ cells in peripheral blood were analyzed by staining them with MHC Class I Murine Tetramer. C57BL/6-Tg mice with a transgenic T-cell receptor designed to recognize ovalbumin epitope, were used as positive controls for OVA-CD8⁺ cells. The data shown in FIGS. 2A and 2B suggest that the administration of AAV6-S663V+T492V vectors expressing OVA led to a robust activation (approximately 9%) of specific T-cells compared to the AAV6-WT-OVA treated animals (less than 1%). The maximum of amplitude of the OVA-CD8⁺ cell number was observed two weeks after i.m. injection. Three weeks after injection, number of OVA specific T-cells was below the detectable level (FIG. 2C). These data indicate that capsid-optimized AAV6 vectors have the potential of being used as an immune modulator more efficiently than the AAV6-WT vectors.

Capsid-Optimized AAV6 Vectors Stimulate Specific T-Cell Clone with a High Killing Ability.

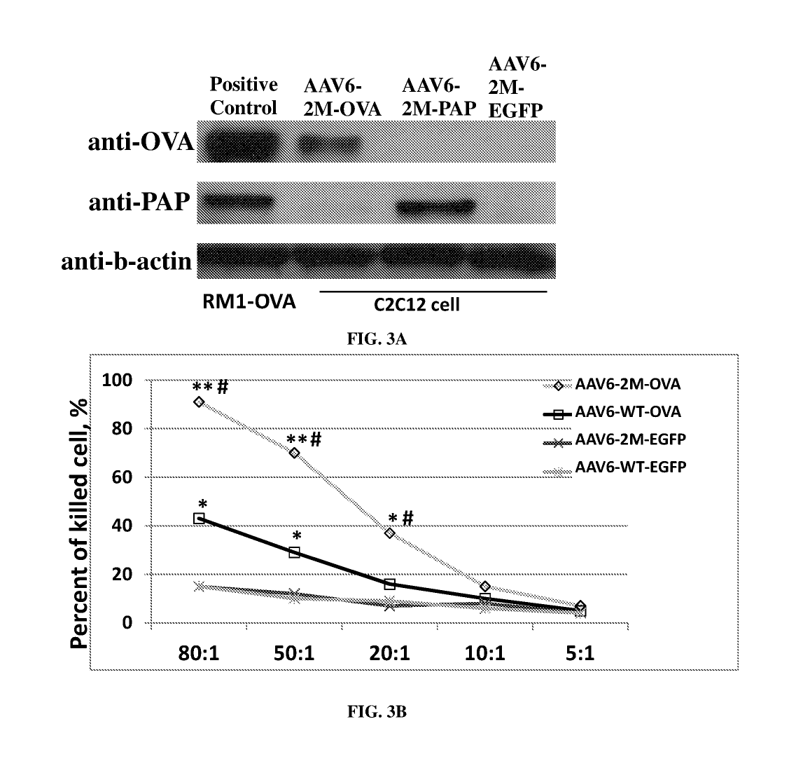

Since the capsid-optimized AAV6 vector-mediated specific T-cells clone proliferation was significantly improved when compared with the AAV6-WT vectors, the cytotoxic ability of these T-cells against cancer cells in vitro was evaluated next. First, the expression level of PAP and OVA proteins following infection of cap sid-optimized AAV6 vectors in a murine myoblast cell line, C2C12, was evaluated and compared them with the naturally occurring expression of PAP and stable expression of OVA in RM1 and RM1-OVA cells (FIG. 3A). AAV6 vectors expressing EGFP were used to rule out possible stimulation of expression during infection. These results indicate a high level of expression of proteins with AAV6 mutant vectors. Then, OVA-CD8⁺ cells were generated from splenocytes from mice i.m. injected with AAV6-WT-OVA and AAV-S663V+T492V-OVA vectors, as described above. AAV6-WT-EGFP and AAV6-S663V+T492V-EGFP vectors were used as appropriate controls. Mouse prostate cancer cells, RM1, stably expressing OVA, were used as a specific target for a two-color fluorescence assay of cell-mediated cytotoxicity to generate a killing curve using reduced effector to target cell ratio, as described above. Results of these experiments, shown in FIG. 3, suggest that i.m. injection of capsid-optimized AAV6 vectors can effectively stimulate specific T-cell clone proliferation and with a ˜4-fold higher killing activity compared with non-specific control and ˜2-fold higher than AAV6-WT vectors. The use of AAV6-WT-EGFP or AAV6-S663V+T492V-EGFP vectors for non-specific control T-cells rules out the possible auto-reactivity or possible non-specific response of stimulated cytotoxicity. Since immunization strategies that generate potent effector responses are essential for effective immunotherapy, the results support the efficacy of capsid-optimized AAV6-based vectors for vaccination studies.

Capsid-Optimized AAV6 Vectors Suppress Tumor Growth and Extend Survival In Vivo.

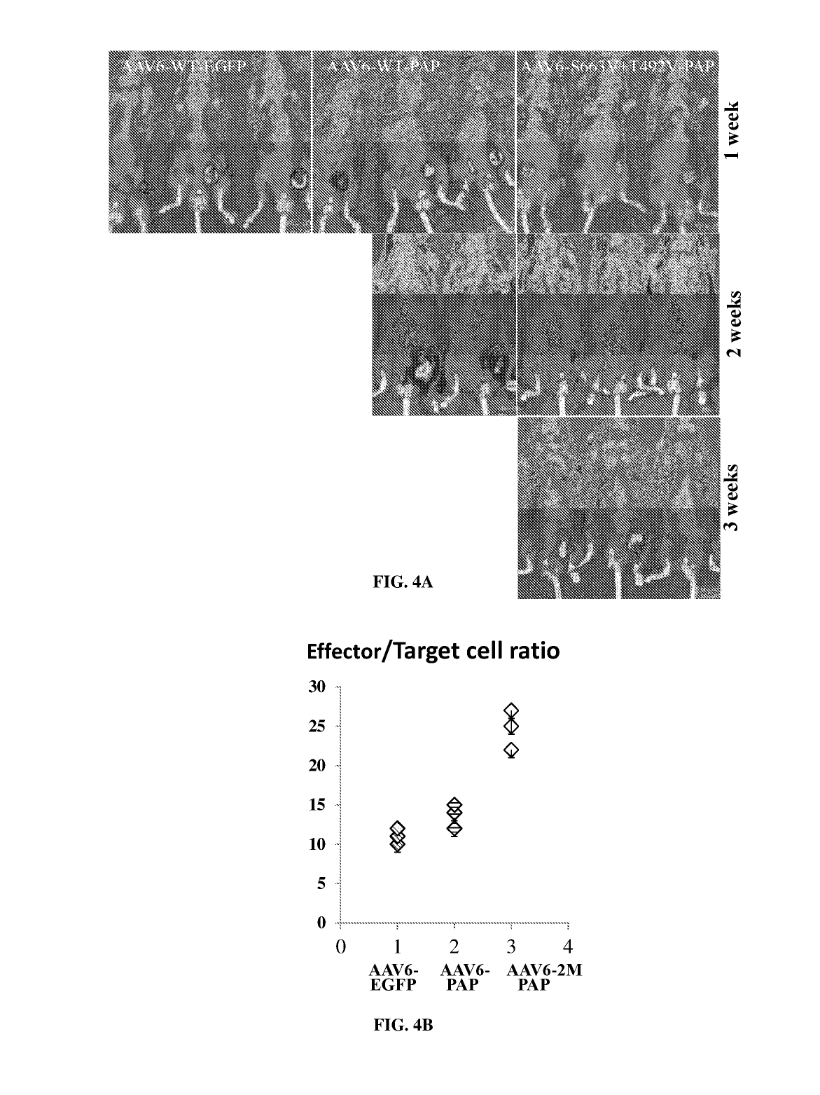

Finally, the ability of cap sid-optimized AAV6 vectors to suppress tumor growth in an animal model was assessed. Prostatic acid phosphatase (PAP), a gene upregulated in both human and mouse prostate cancer, was used as a specific target in vivo. Three groups of three C57BL/6 mice (n=3) were i.m. injected with either the AAV6-S663V+T492V-PAP, AAV6-WT-PAP or AAV6-WT-EGFP vectors. Two weeks post viral injection, when the number of specific T-cells reached maximum amplitude, as was evaluated previously, mice were challenged with the prostate cancer cell line, RM1, stably expressing the firefly luciferase (RM1-FLuc), by subcutaneous injection. Tumor growth was then measured weekly by bioluminescence imaging (FIG. 4A). Mice injected with AAV6-WT vectors containing EGFP were sacrificed one week after cancer cell challenge according to body score condition recommended by the Institutional Animal Care and Use Committee (IACUC). However, mice injected with AAV6-WT-PAP survived over two weeks. A significant suppression of tumor growth was observed in the group of animals injected with the capsid-optimized AAV6-S663V+T492V vectors expressing PAP. The life span of two mice was extended for about three weeks, and for the third mouse, up to four weeks (FIG. 4B). The results clearly indicate the ability of the mutant AAV6-S663V+T492V-PAP vectors to suppress tumor growth for a longer duration of time compared with the AAV6-WT vectors, containing either a cancer specific (PAP) or the reporter (EGFP) gene.

Recent developments of preclinical and clinical gene therapy protocols have demonstrated favorable safety profile and long-term gene expression by AAV vectors (11, 30, 31). However, generally low immunogenicity of commonly used serotypes of AAV can likely explain the lack of interest to employ these vectors for immunization studies (32, 33). Currently, more than a hundred different AAV variants have been isolated from human and non-human tissues, but only a few have been used as recombinant vectors. Selection of these AAV-based vectors was determined mostly by specific tissue-tropism and transduction efficiency. However, several studies have documented significant differences in immune response induced by AAV depending on the capsid composition and the route of vector administration (32, 34-37). Thus, the natural plasticity of AAV warrants discovering or developing variants capable of stimulating stronger immune responses (28, 29, 38, 39). For example, a recently identified AAV/rh32.33 serotype, has a unique capsid structure that elicits cellular immune response toward vector encoded antigens (40). An even greater possibility to identify a superior AAV vector for the manipulation of host immune response was offered by bioengineering of the viral capsid using recombinant libraries or by inserting an immunogenic peptide on the capsid surface (41-43). It was also shown that naturally occurring serotypes of AAV vectors are not optimal for the transduction of a number of cell types, including DCs, and that the efficacy of these vectors can be significantly enhanced by mutation of critical surface-exposed serine and threonine residues on their capsids. Previously published data indicate that such optimization of AAV2 and AAV6 leads to high activity of these vectors in human monocyte-derived dendritic cells (moDCs) (14, 15). This increase in infectivity is associated with advanced nuclear translocation rather than improved viral entry into cells. Another significant advantage of the employment of such capsid-modified AAV vectors for targeting DCs is that escape from the proteasomal pathway provides less material for potential presentation, and prevents induction of immune responses against vector-derived epitopes. Indeed, it was shown that such engineered AAV vectors minimize in vivo targeting of transduced hepatocytes by capsid-specific CD8+ T-cells (44). Also, considering the relatively short period of time for cytopathic changes in DCs transduced by AAV vectors, early onset of antigen expression is critical for appropriate presentation, and consequentially, T-cell activation.

The time of T-cell activation and the potency and longevity of responses are crucial factors in evaluation of the possible therapeutic outcomes. Thus, whether an increased transduction efficiency of DCs by the capsid-optimized AAV6-S663V+T492V vectors correlated with superior priming of T-cells was also evaluated. The results suggest that modification of the AAV6 capsid is beneficial in terms of producing a robust antigen-specific peripheral T-cell response. Moreover, vaccination via intramuscular injection of the capsid-optimized AAV6-S663V+T492V vectors encoding a specific antigen (PAP) induces a protective immune response which leads to significant delay in prostate cancer progression and extends survival in mice.

In some embodiments, an even more efficient immune response can be induced. For example, in some embodiments, a novel AAV serotype with immunogenic properties can be used. In some embodiments, the effectiveness of the immune response can be increased by using adjuvants, such as unmethylated CpG oligodinucleotide, granulocyte-macrophage colony-stimulating factor (GM-CSF) or interleukin 12 (Il-12)(45), or other adjuvants. Transient activation of toll-like receptors 9 (TLR9) followed by AAV infection was previously described (46). Thus, a selective TLR9 agonist also can be used along with AAV administration to enhance immune response (47).

Accordingly, the current studies provide support for the use of capsid-optimized AAV vectors for immunomodulation in general, and for inducing a protective anti-cancer immune response in particular.

### REFERENCES

1. Banchereau J, Steinman R M. Dendritic cells and the control of immunity. Nature. 1998; 392(9521319):245-52.
2. Figdor C G, de Vries I J, Lesterhuis W J, Melief C J. Dendritic cell immunotherapy: mapping the way. Nat Med. 2004; 10(5):475-80.
3. Palucka K, Ueno H, Banchereau J. Recent developments in cancer vaccines. J Immunol. 2011; 186(21248270):1325-31.
4. Ueno H, Schmitt N, Klechevsky E, Pedroza-Gonzalez A, Matsui T, Zurawski G, et al. Harnessing human dendritic cell subsets for medicine. Immunol Rev. 2010; 234(20193020):199-212.
5. Hoos A, Britten C M, Huber C, O'Donnell-Tormey J. A methodological framework to enhance the clinical success of cancer immunotherapy. Nat Biotechnol. 2011; 29(21997622):867-70.
6. Humbert J M, Halary F. Viral and non-viral methods to genetically modify dendritic cells. Curr Gene Ther. 2012; 12(2):127-36.
7. Tacken R I, de Vries I J M, Torensma R, Figdor C G. Dendritic-cell immunotherapy: from ex vivo loading to in vivo targeting. Nat Rev Immunol. 2007; 7(17853902):790-802.
8. Ueno H, Klechevsky E, Schmitt N, Ni L, Flamar A-L, Zurawski S, et al. Targeting human dendritic cell subsets for improved vaccines. Semin Immunol. 2011; 23(21277223):21-7.
9. Robert-Guroff M. Replicating and non-replicating viral vectors for vaccine development. Curr Opin Biotechnol. 2007; 18(6):546-56.
10. Daya S, Berns K I. Gene therapy using adeno-associated virus vectors. Clin Microbiol Rev. 2008; 21(18854481):583-93.
11. High K A, Aubourg P. rAAV human trial experience. Methods Mol Biol. 2011; 807:429-57.
12. Srivastava A. Adeno-associated virus-mediated gene transfer. J Cell Biochem. 2008; 105(18500727):17-24.
13. Ling C Q, Wang L N, Wang Y, Zhang Y H, Yin Z F, Wang M, et al. The roles of traditional Chinese medicine in gene therapy. J Integr Med. 2014; 12(2):67-75.
14. Aslanidi G V, Rivers A E, Ortiz L, Govindasamy L, Ling C, Jayandharan G R, et al. High-efficiency transduction of human monocyte-derived dendritic cells by capsid-modified recombinant AAV2 vectors. Vaccine. 2012; 30(22497875):3908-17.
15. Pandya J, Ortiz L, Ling C, Rivers A E, Aslanidi G. Rationally designed capsid and transgene cassette of AAV6 vectors for dendritic cell-based cancer immunotherapy. Immunol Cell Biol. 2013.
16. Ponnazhagan S, Mahendra G, Curiel D T, Shaw D R. Adeno-associated virus type 2-mediated transduction of human monocyte-derived dendritic cells: implications for ex vivo immunotherapy. J Virol. 2001; 75(11533211):9493-501.
17. Shin O, Kim S J, Lee W I, Kim J Y, Lee H. Effective transduction by self-complementary adeno-associated viruses of human dendritic cells with no alteration of their natural characteristics. J Gene Med. 2008; 10(18452239):762-9.
18. Ussher J E, Taylor J A. Optimized transduction of human monocyte-derived dendritic cells by recombinant adeno-associated virus serotype 6. Hum Gene Ther. 2010; 21(20578847):1675-86.
19. Veron P, Allo V, Riviere C, Bernard J, Douar A-M, Masurier C. Major subsets of human dendritic cells are efficiently transduced by self-complementary adeno-associated virus vectors 1 and
2. J Virol. 2007; 81(17314166):5385-94.
20. Balazs A B, Bloom J D, Hong C M, Rao D S, Baltimore D. Broad protection against influenza infection by vectored immunoprophylaxis in mice. Nat Biotechnol. 2013; 31(7):647-52. PMCID: 4030719.
21. Deal C, Balazs A B, Espinosa D A, Zavala F, Baltimore D, Ketner G. Vectored antibody gene delivery protects against Plasmodium falciparum sporozoite challenge in mice. Proc Natl Acad Sci USA. 2014; 111(34):12528-32. PMCID: 4151717.
22. Eisold S, Schmidt J, Ryschich E, Gock M, Klar E, von Knebel Doeberitz M, et al. Induction of an antitumoral immune response by wild-type adeno-associated virus type 2 in an in vivo model of pancreatic carcinoma. Pancreas. 2007; 35(17575547):63-72.
23. Mahadevan M, Liu Y, You C, Luo R, You H, Mehta J L, et al. Generation of robust cytotoxic T lymphocytes against prostate specific antigen by transduction of dendritic cells using protein and recombinant adeno-associated virus. Cancer Immunol Immunother. 2007; 56(17356843):1615-24.
24. Nieto K, Salvetti A. AAV Vectors Vaccines Against Infectious Diseases. Front Immunol. 2014; 5:5. PMCID: 3896988.
25. Nieto K, Stahl-Hennig C, Leuchs B, Muller M, Gissmann L, Kleinschmidt J A. Intranasal vaccination with AAVS and 9 vectors against human papillomavirus type 16 in rhesus macaques. Hum Gene Ther. 2012; 23(7):733-41. PMCID: 3404423.
26. Yu Y, Pilgrim P, Zhou W, Gagliano N, Frezza E E, Jenkins M, et al. rAAV/Her-2/neu loading of dendritic cells for a potent cellular-mediated MHC class I restricted immune response against ovarian cancer. Viral Immunol. 2008; 21(19115932):435-42.
27. Aslanidi G V, Rivers A E, Ortiz L, Song L, Ling C, Govindasamy L, et al. Optimization of the capsid of recombinant adeno-associated virus 2 (AAV2) vectors: the final threshold? PLoS One. 2013; 8(3):e59142. PMCID: 3602601.
28. Zhong L, Li B, Jayandharan G, Mah C S, Govindasamy L, Agbandje-McKenna M, et al. Tyrosine-phosphorylation of AAV2 vectors and its consequences on viral intracellular trafficking and transgene expression. Virology. 2008; 381(18834608):194-202.
29. Zhong L, Li B, Mah C S, Govindasamy L, Agbandje-McKenna M, Cooper M, et al. Next generation of adeno-associated virus 2 vectors: point mutations in tyrosines lead to high-efficiency transduction at lower doses. Proc Natl Acad Sci USA. 2008; 105(22):7827-32. PMCID: 2402387.
30. Asokan A, Schaffer D V, Samulski R J. The AAV vector toolkit: poised at the clinical crossroads. Mol Ther. 2012; 20(22273577):699-708.
31. Flotte T R, Trapnell B C, Humphries M, Carey B, Calcedo R, Rouhani F, et al. Phase 2 clinical trial of a recombinant adeno-associated viral vector expressing alphal-antitrypsin: interim results. Hum Gene Ther. 2012; 22(10):1239-47.
32. Mingozzi F, High K A. Immune responses to AAV in clinical trials. Curr Gene Ther. 2007; 7(5):316-24.
33. Mueller C, Chulay J D, Trapnell B C, Humphries M, Carey B, Sandhaus R A, et al. Human Treg responses allow sustained recombinant adeno-associated virus-mediated transgene expression. J Clin Invest. 2013; 123(12):5310-8. PMCID: 3859421.
34. Lin J, Calcedo R, Vandenberghe L H, Bell P, Somanathan S, Wilson J M. A new genetic vaccine platform based on an adeno-associated virus isolated from a rhesus macaque. J Virol. 2009; 83(24):12738-50. PMCID: 2786857.
35. Lin J, Zhi Y, Mays L, Wilson J M. Vaccines based on novel adeno-associated virus vectors elicit aberrant CD8+ T-cell responses in mice. J Virol. 2007; 81(21):11840-9. PMCID: 2168776.
36. Nieto K, Kern A, Leuchs B, Gissmann L, Muller M, Kleinschmidt J A. Combined prophylactic and therapeutic intranasal vaccination against human papillomavirus type-16 using different adeno-associated virus serotype vectors. Antivir Ther. 2009; 14(8):1125-37.
37. Vandenberghe L H, Wilson J M. AAV as an immunogen. Curr Gene Ther. 2007; 7(17979679):325-33.
38. Li C, Diprimio N, Bowles D E, Hirsch M L, Monahan P E, Asokan A, et al. Single amino acid modification of adeno-associated virus capsid changes transduction and humoral immune profiles. J Virol. 2012; 86(15):7752-9. PMCID: 3421647.
39. Wu P, Xiao W, Conlon T, Hughes J, Agbandje-McKenna M, Ferkol T, et al. Mutational analysis of the adeno-associated virus type 2 (AAV2) capsid gene and construction of AAV2 vectors with altered tropism. J Virol. 2000; 74(10954565):8635-47.
40. Mays L E, Vandenberghe L H, Xiao R, Bell P, Nam H-J, Agbandje-McKenna M, et al. Adeno-associated virus capsid structure drives CD4-dependent CD8+ T cell response to vector encoded proteins. J Immunol. 2009; 182(19414756):6051-60.
41. Bartel M A, Weinstein J R, Schaffer D V. Directed evolution of novel adeno-associated viruses for therapeutic gene delivery. Gene Ther. 2012; 19(6):694-700.
42. Vandenberghe L H, Wilson J M, Gao G. Tailoring the AAV vector capsid for gene therapy. Gene Ther. 2009; 16(3):311-9.
43. Nieto K, Weghofer M, Sehr P, Ritter M, Sedlmeier S, Karanam B, et al. Development of AAVLP(HPV16/31L2) particles as broadly protective HPV vaccine candidate. PLoS One. 2012; 7(6):e39741. PMCID: 3384601.
44. Martino A T, Basner-Tschakarjan E, Markusic D M, Finn J D, Hinderer C, Zhou S, et al. Engineered AAV vector minimizes in vivo targeting of transduced hepatocytes by capsid-specific CD8+ T cells. Blood. 2013; 121(12):2224-33. PMCID: 3606062.
45. van de Laar L, Coffer R I, Woltman A M. Regulation of dendritic cell development by GM-CSF: molecular control and implications for immune homeostasis and therapy. Blood. 2012; 119(15):3383-93.
46. Jayandharan G R, Aslanidi G, Martino A T, Jahn S C, Perrin G Q, Herzog R W, et al. Activation of the NF-kappaB pathway by adeno-associated virus (AAV) vectors and its implications in immune response and gene therapy. Proc Natl Acad Sci USA. 2011; 108(9):3743-8. PMCID: 3048154.
47. Triozzi P L, Aldrich W, Ponnazhagan S. Inhibition and promotion of tumor growth with adeno-associated virus carcinoembryonic antigen vaccine and Toll-like receptor agonists. Cancer Gene Ther. 2011; 18(12):850-8.
48. Zhang Y H, Wang Y, Yusufali A H, Ashby F, Zhang D, Yin Z F, et al. Cytotoxic genes from traditional Chinese medicine inhibit tumor growth both in vitro and in vivo. J Integr Med. 2014; 12(6):483-94.
49. Zolotukhin S, Potter M, Zolotukhin I, Sakai Y, Loiler S, Fraites T J, et al. Production and purification of serotype 1, 2, and 5 recombinant adeno-associated viral vectors. Methods. 2002; 28(12413414):158-67.
50. Mattis A E, Bernhardt G, Lipp M, Forster R. Analyzing cytotoxic T lymphocyte activity: a simple and reliable flow cytometry-based assay. J Immunol Methods. 1997; 204(9212830):135-42.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A recombinant adeno-associated virus (rAAV) particle comprising a nucleic acid that encodes a cancer-associated antigen under the control of a promoter.

2. The rAAV particle of claim 1, wherein the nucleic acid is an expression construct that is flanked on each side by an inverted terminal repeat sequence.

3. The rAAV particle of claim 1, wherein the cancer associated antigen is Prostatic Acid Phosphatase (PAP), Prostate specific antigen (PSA), Prostate-specific membrane antigen (PSMA), Cancer Antigen 15-3 (CA-15.3), Epidermal growth factor receptor 2 (Her2/neu), FMS-like tyrosine kinase 3 ligand (FLT3), Alpha-fetoprotein (AFP), Hepatocyte growth factor receptor (HGFR, c-Met), Glypican 3 (GLP3), Carcinoembryonic antigen (CEA), and/or Telomerase (TERT).

4. The rAAV particle of claim 1, wherein the nucleic acid is a single-stranded or self-complementary rAAV nucleic acid vector.

5. The rAAV particle of claim 1, wherein the rAAV particle is an rAAV6 particle.

6. The rAAV particle of claim 1, wherein the rAAV6 particle comprises a modified capsid protein comprising a non-native amino acid substitution at a position that corresponds to a surface-exposed amino acid in a wild-type AAV6 capsid protein.

7. The rAAV particle of claim 6, wherein the non-native amino acid substitution is selected from a non-tyrosine amino acid at a wild-type tyrosine position, a non-serine amino acid at a wild-type serine position, a non-threonine amino acid at a wild-type threonine position, a non-lysine amino at a wild-type lysine position, or a combination thereof.

8. The rAAV particle of claim 7, comprising a Valine at position S663.

9. The rAAV particle of claim 7, comprising a Valine at position T492.

10. The rAAV particle of claim 7, comprising a Valine at position S662 and a Valine at position T492.

11. A composition comprising a plurality of different rAAV particles of claim 1.

12. A composition comprising a rAAV particle of claim 1 and further comprising a pharmaceutically acceptable carrier.

13. A composition comprising a rAAV particle of claim 1 and further comprising an adjuvant.

14. The composition of claim 13, wherein the adjuvant is an unmethylated CpG oligodinucleotide, a granulocyte-macrophage colony-stimulating factor (GM-CSF), interleukin 12 (11-12), or an agonist of a toll-like receptor 9 (TLR9).

15. A method of providing an immunotherapy for treating cancer in a subject, the method comprising delivering an rAAV particle or composition of claim 1 to a subject in an amount sufficient to produce an immunotherapeutic response in the subject.

16. The method of claim 15, further comprising delivering an adjuvant to the subject.

17. The method of claim 15, further comprising delivering an adjuvant therapy to the subject.

18. The method of claim 15, wherein the rAAV particle or composition is injected subcutaneously, intramuscularly, or intradermally.

19. The method of claim 15, wherein the subject was diagnosed as having cancer.

20. The method of claim 15, wherein the subject is at risk of developing cancer.

21. The method of claim 15, wherein the cancer is selected from the group consisting of lymphomas, hemangiosarcomas, mast cell tumors, osteosarcomas, melanomas, prostate cancer, thyroid cancer, liver cancer, pancreatic cancer, brain tumors, kidney cancer, ocular cancer, head or neck cancer, lung cancer, breast cancer, cervical cancer, gastrointestinal cancers, and urogenital cancers.

22. The method of claim 15, wherein the subject is a mammal.

23. The method of claim 22, wherein the subject is a human, a non-human primate, a companion animal, or a farm animal.

24. The method of claim 17, further comprising delivering a chemotherapeutic agent to the subject.

25. The method of claim 15, further comprising delivering an additional immunotherapeutic agent to the subject before delivering the rAAV particle or composition.

26. The additional immunotherapeutic agent of claim 25, wherein the immunotherapeutic agent is a DNA plasmid vector containing a cancer associated antigen.

27. The method of claim 15, further comprising delivering an additional immunotherapeutic agent to the subject after delivering the rAAV particle or composition.

28. The additional immunotherapeutic agent of claim 27, wherein the immunotherapeutic agent is a DNA plasmid vector containing a cancer associated antigen.


SEQUENCE LISTING (5 sequences)

SEQ ID NO: 1 - protein, 736 aa, Adeno-associated virus serotype 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYFLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, Adeno-associated virus serotype 2 [sequence omitted]

SEQ ID NO: 3 - DNA, 21 bp, Artificial Sequence
  Synthetic Polynucleotide
tcccatagtaacgccaatagg

SEQ ID NO: 4 - DNA, 23 bp, Artificial Sequence
  Synthetic Polynucleotide
cttggcatatgatacacttgatg

SEQ ID NO: 5 - protein, 8 aa, Artificial Sequence
  Synthetic Polypeptide
SIINFEKL